(19) United States
(12) Patent Application Publication
(10) Pub. No.: US 2011/0137041 A1
(43) Pub. Date: Jun. 9, 2011

(54) PROCESS FOR PREPARING ATOVAQUONE AND ASSOCIATE INTERMEDIATES

(75) Inventors: Fuqiang Zhu, Shanghai (CN); He Qiao, Shanghai (CN); Michel Bekhazi, Pointe Claire (CA)

(73) Assignee: CHEMAGIS LTD., Bnei Brak (IL)

(21) Appl. No.: 13/001,159

(22) PCT Filed: Jun. 30, 2008
(86) PCT No.: PCT/IL2008/000893

§ 371 Date: Feb. 24, 2011

(51) Int. Cl. C07D 213/60; C07D 213/71; C07C 45/00

(52) U.S. Cl. 546/290; 546/296; 568/309

(57) ABSTRACT

The invention provides novel intermediates of atovaquone and use thereof for the preparation of atovaquone

### FIELD OF THE INVENTION

The invention relates to novel intermediates of atovaquone and to an improved process for preparing atovaquone.

### BACKGROUND OF THE INVENTION

Atovaquone, trans-(2-[4-(4-chlorophenyl)cyclohexyl]-3-hydroxy-1,4-naphthoquinone (compound I), is represented by the following structural formula:

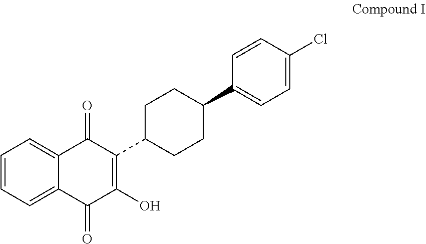

Atovaquone is the active ingredients in two drugs which are marketed in the United State, Europe and other countries by GSK. The first drug is an oral suspension (750 mg/5 mL) under the trade name Mepron® which is indicated for the treatment and prophylaxis of Pneumocystis carinii infection. The second drug is a combination with proguanil hydrochloride, under the brand name Malarone® for the prophyaxis of Malaria. Malaron® is supplied as an oral tablet containing 250 mg of atovaquone and 100 mg of proguanil hydrochloride and a pediatric dosage containing 62.5 mg of atovaquone and 25 mg of Proguanil hydrochloride.

The synthesis of atovaquone was disclosed in U.S. Pat. No. 4,981,874, herein referred to as the '874 patent. The process is illustrated in scheme 1.

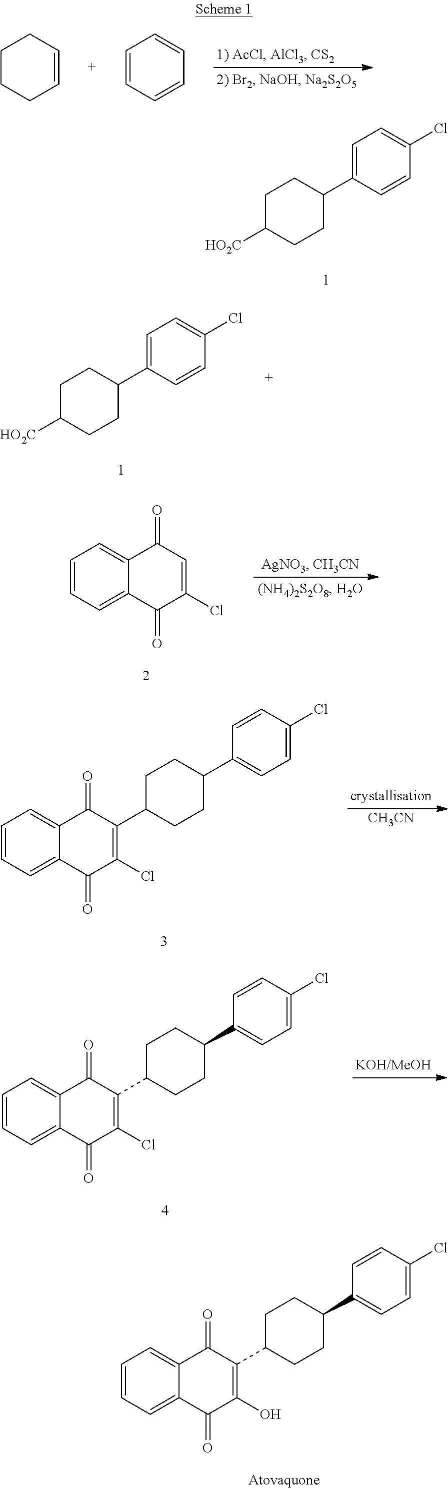

The process described in the 874' patent is reported to give a low yield of atovaquone (4% total yield of atovaquone calculated from the last two steps).

An additional process is disclosed in Tetrahedron Letters 39 (1998) 7629-7632 (David R. Williams and Michael P. Clark). The mixture of cis and trans isomers of formula 3 are produced by reacting the oxalate of formula 5, with 2-chloro-1,4-naphthquinone, a compound of formula 2, in the presence of silver nitrate, ammonium per sulphate and a phase transfer catalyst such as Adogen 464. The crude produced is purified by flash chromatography using ethyl acetate/hexanes to isolate 2-[4-(4-chlorophenyl)cyclohexyl]-3-chloro-1,4-naphtoquinone, compound of formula 3 (ratio of trans/cis-isomers 1.3 to 1, 43% yield) and the ester by-product, 3-chloro-1,4-dihydro-1,4-dioxo-4-(4-chlorophenyl)cyclohexyl ester-2-naphthalencarboxylic acid of formula 6 (38% yield). Finally the conversion to atovaqoune was performed as described in the '874 patent mentioned above. The process is illustrated in scheme 2.

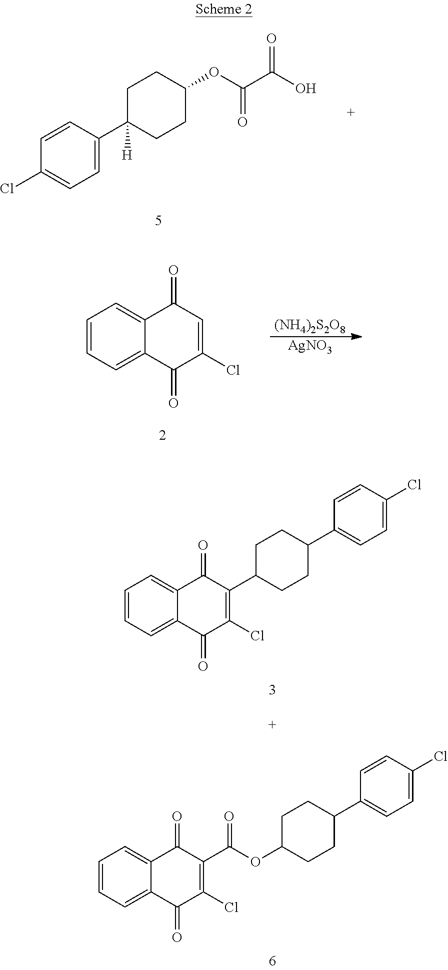

The disadvantage of the above process is that the resulting product 3 is purified by column chromatography, which is time, money and solvents consuming and difficult to apply in industrial large scale production. Further more the next step of the conversion to atovaquone is expected to provide low yield as described in the 874' patent.

The processes described above are reported to give low yields of atovaquone. Those processes further include silver nitrate (a heavy metal) which is expensive and may contaminate the final product with silver, tightly controlled by health authorities and might be difficult to remove. There is an unmet need for an improved process which provides higher yields of pure atovaquone, using reagents which are unexpensive while avoiding the use of heavy metals. The present invention provides such a process.

### SUMMARY OF THE INVENTION

The invention provides novel intermediates, compounds (IV) and (V), and uses thereof for preparing atovaquone.

The process for preparing atovaquone comprising:
(a) reacting 4-(4-chlorophenyl)cyclohexane-1-carboxylic acid of formula (II) with N-hydroxypyridine-2-thione of formula (III), in the presence of an esterification reagent, to form 2-thioxopyridin-1(2H)-yl-4-(4-chlorophenyl)-cyclohexane carboxylate, compound of formula (IV);

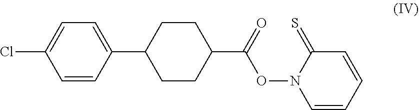

(b) reacting compound (IV) with 1,4-napthoquinone to form 2-[4-(4-chlorophenyl)cyclohexyl]-3-(2-pyridin-2-ylthio)-naphthalene-1,4-dione, the compound of formula (V);

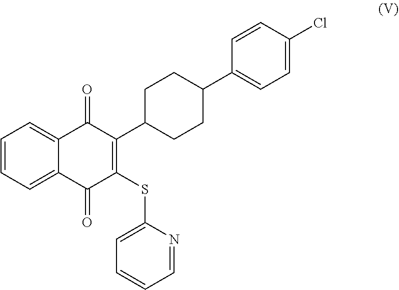

(c) converting the compound of formula (V) into 2-[4-(4-chlorophenyl)cyclohexyl]-3-hydroxy-1,4-naphthoquinone of formula (VI) in the presence of a base; and
(d) isolating the trans 2-[4-(4-Chlorophenyl)cyclohexyl]-3-hydroxy-1,4-naphthoquinone of formula (I).

### DETAILED DESCRIPTION OF THE INVENTION

The invention provides novel intermediates, compounds (IV) and (V), and uses thereof for preparing atovaquone, as depicted in scheme 3. The process for preparing atovaquone comprising:
(a) reacting 4-(4-chlorophenyl)cyclohexane-1-carboxylic acid of formula (II) with N-hydroxypyridine-2-thione of formula (III) in the presence of an esterification reagent, to form 2-thioxopyridin-1(2H)-yl-4-(4-chlorophenyl)-cyclohexane carboxylate, compound of formula (IV);

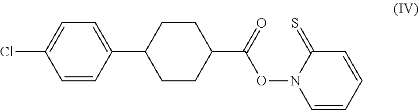

(b) reacting compound (IV) with 1,4-napthoquinone to form 2-[4-(4-chlorophenyl)cyclohexyl]-3-(2-pyridin-2-ylthio)-naphthalene-1,4-dione, the compound of formula (V);

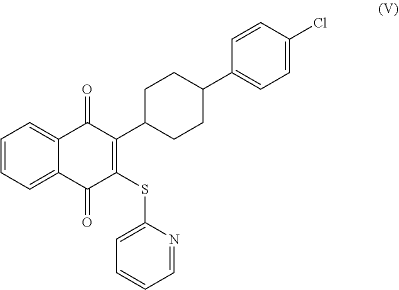

(c) converting the compound of formula (V) into 2-[4-(4-chlorophenyl)cyclohexyl]-3-hydroxy-1,4-naphthoquinone of formula (VI) in the presence of a base; and
(d) isolating the trans 2-[4-(4-Chlorophenyl)cyclohexyl]-3-hydroxy-1,4-naphthoquinone of formula (I).

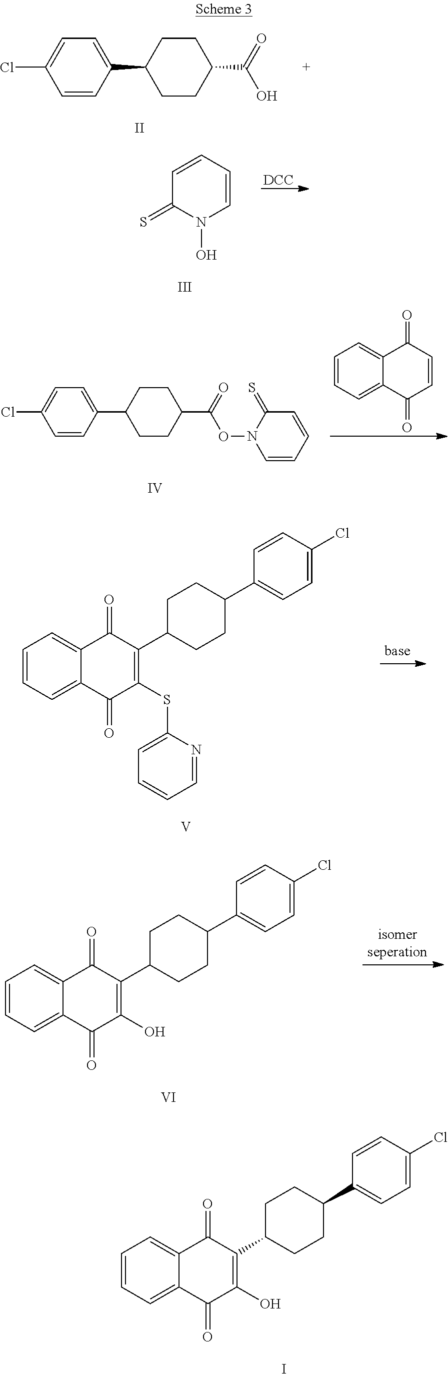

According to the present invention step (a) includes:

admixing 4-(4-chlorophenyl)cyclohexane-1-carboxylic acid of formula (II) with N-hydroxypyridine-2-thione of formula (III) in an organic solvent,

cooling to reduce the temperature, adding an esterification reagent, optionally in several portions, and isolating compound (IV).

In accordance with the present invention isolating compound (IV) further comprises:

(i) filtering the reaction mixture;

(ii) evaporating a portion of the solvent,

(iii) adding a non polar anti solvent;

(iv) collecting the product by filtration, washing and drying.

Suitable non limiting examples of organic solvents for the reaction of step (a) include: dichloromethane, dichloroethane, chloroform, acetonitrile, tetrahydrofuran (THF), acetone, dioxane or a mixture thereof. A preferred organic solvent is dichloromethane.

Suitable non limiting examples of esterification reagents include: dicyclohexylcarbodiimide (DCC), 3-dimethylaminopropyl carbodiimide (EDC), diisopropylcarbodiimide (DIC). A preferred esterification reagent is DCC.

Suitable non limiting examples of non polar anti solvent include: heptane, cyclohexane, petroleum ether, hexane, preferably petroleum ether.

The process of obtaining compound (IV) may be carried out in a temperature range of −5° C. to 15° C., preferably at 0-5° C.

Preferably, the molar ratio between compound (II), compound (III) and the esterification reagent (e.g DCC) is 1:1:1.

According to the present invention step (b) includes:

irradiating compound (IV) with 1,4-napthoquinone in an organic solvent; and isolating the obtained compound (IV).

It has been found that the isomeric configuration (e.g cis, trans or mixture thereof) of compound (IV) is lost during the reaction of step (b) and the thus formed compound (v) is a mixture of cis and trans.

In accordance with the present invention the isolation of compound (IV) further comprises:

(i) concentrating the mixture,

(ii) adding a polar organic solvent and stirring the mixture at elevated temperature;

(iii) filtering the obtained compound (V), washing, drying, and optionally

(iv) purifying the obtained compound (V).

Suitable non limiting examples of organic solvents for the reaction of step (b) include: dichloromethane, dichloroethane, chloroform, carbon tetrachloride, toluene, acetonitrile and mixture thereof. A preferred solvent for the reaction is dichloromethane.

Suitable non limiting examples of a polar organic solvent include: methanol, ethanol, 1-propanol, 2-propanol, butanol, and mixture thereof. A preferred solvent is ethanol.

Preferably, the molar ratio of compound (IV) to the 1,4-naphtoquinone is 1:2.

The reaction of step (b) may be carried out in a temperature range of −5° C. to 15° C., preferably at 0-5° C. and the reaction mixture may be irradiated in the visible spectrum from 380 to 750 nm. Preferably, the irradiation is carried out by a 400 W halogen lamp.

In accordance with the present invention the mixture is stirred with a polar organic solvent at a temperature range of 35-65° C., preferably at 45-55° C.

Compound (V) may be purified by slurring the obtained solid in a polar organic solvent, optionally at elevated temperature; and collecting the product by filtration. Compound (V) may also be purified by recrystallization from an organic solvent.

Suitable non limiting examples of organic solvents for the recrystallization of compound (V) includes: methanol, ethanol, propanol, isopropanol, n-butanol, acetonitrile, ethyl acetate, acetone and mixture thereof, preferably acetonitrile.

Suitable non limiting examples of organic solvents for slurring compound (V) include: methanol, ethanol, propanol, isopropanol, n-butanol, acetonitrile, ethyl acetate, acetone and mixture thereof, preferably ethanol.

According to the present invention step (c) comprises:

reacting compound (V) with a base in a polar organic solvent at elevated temperatures.

In accordance with the present invention step (c) of reacting compound (V) with a base further comprises:

(i) admixing compound (V) with a polar organic solvent,

(ii) adding a base dissolved in water, optionally dropwise,

(iii) stirring at elevated temperatures,

(iv) extracting the reaction mixture with a non polar organic solvent,

(v) separating the phases and acidifying the aqueous layer with an acid.

Suitable non limiting examples of a polar organic solvent include: methanol (MeOH), ethanol (EtOH), 1-propanol, 2-propanol, dimethylformamide (DMF), or mixture thereof, preferably methanol.

Suitable non limiting examples of bases include: sodium hydroxide, potassium hydroxide, lithium hydroxide, sodium carbonate, potassium phosphate, sodium phosphate and sodium bicarbonate. A preferred base is sodium hydroxide.

Suitable non limiting examples of non polar organic solvents include: hexane, heptane, cyclohexane, petroleum ether, diethyl ether, diisopropyl ether, methyl t-butyl ether and mixtures thereof. A preferred organic solvent is heptane.

Suitable non limiting examples of acids can include inorganic acids selected from: HCl and sulfuric acid.

The molar ratio of the base to compound (IV) may be from 1:1 to 10:1, preferably 6:1.

The temperature range for stirring the reaction mixture may be from 50 to 65° C., preferably at 55-60° C.

According to the present invention the isolation of compound (I), step (d) comprises:

collecting the solid obtained by filtration, washing, drying, and optionally recrystallizing the crude product from an organic solvent or mixture of organic solvents.

Suitable non limiting examples of organic solvents are: THF, acetone, acetonitrile, dioxane, ethanol, methanol, ethyl acetate, methyl acetate, and combination thereof. Preferably, the solvent used for crystallizing compound (I) is acetonitrile

In a specific embodiment of the present invention step (a) includes admixing 4-(4-chlorophenyl)cyclohexane-1-carboxylic acid of formula (II) with N-hydroxypyridine-2-thione of formula (III) (1:1 ratio) in dichloromethane, cooling to 0-5° C., adding DCC (1 equivalent) portion-wise and stirring. The isolation of compound (IV) includes filtering the reaction mixture; evaporating a portion of the dichloromethane, adding petroleum ether, collecting the product by filtration, washing and drying.

Step (b) includes: irradiating compound (IV) (1 equivalent) with 1,4-napthoquinone (2 equivalents) by a 400 W halogen lamp, in dichloromethane at 0-5° C., concentrating the mixture, adding ethanol and stirring the mixture at 45-55° C.; filtering the obtained compound (V), and further reacting compound (V) (1 equivalent) with sodium hydroxide (6 equivalents) in methanol at 55-60° C., extracting the reaction mixture with heptane; separating the phases, acidifying the aqueous layer with HCl, collecting the solid obtained by filtration, washing, drying, and recrystallizing the crude product from acetonitrile to obtain the pure compound (I).

### Example 1

A 1000 ml 3-necked flask equipped with a thermometer, a dropping funnel and a magnetic stirrer was charged with trans-4-(4-chlorophenyl)cyclohexane-1-carboxylic acid (50 g, 0.21 mol), N-hydroxypyridine-2-thione (26.6 g, 0.21 mol) and dichloromethane (500 mL). DCC (43.2 g, 0.21 mol) was added portion-wise to the mixture at 0-5° C. The mixture was stirred for 3 hours at 0-5° C., then filtered. The obtained solid was stirred with dichloromethane (100 mL) and filtered. The combined organic filtrates were concentrated to about 100 mL and petroleum ether was added (100 mL). The mixture was stirred at 15-20° C. for 30 minutes. The obtained solid was filtered and dried in vacuum to give trans-2-thioxopyridin-1(2H)-yl-4-(4-chlorophenyl)-cyclohexanecarboxylate (compound IV), (87.8% yield), m.p 153-156° C. ¹³C-NMR (CDCl₃) 32.8, 29.0, 42.6, 40.8, 112.6, 128.0, 128.5, 131.8, 133.5, 137.5, 137.6, 144.7, 171.0, 175.8. IR (cm⁻¹) 2929, 1791, 1604, 1527.

### Example 2

Compound (IV) (10 g, 28.7 mmol) and 1,4-napthoquinone (9 g, 57.4 mmol) were added into dichloromethane (100 ml). The mixture was cooled to 0-5° C. and irradiated by a 400 W halogen lamp. After stirring for 40 minutes (reaction completion was monitored by TLC), the crude mixture was concentrated below 35° C., then ethanol (150 mL) was added and the mixture was stirred for 3 hours at 45-55° C. The resulting solid was filtered, washed with ethanol (8 mL) and dried at 50° C. to give 10.6 gr of 2-[4-(4-chlorophenyl)cyclohexyl]-3-(2-pyridin-2-ylthio)-naphthalene-1,4-dione (compound V), (80.2% yield, 48:38 ratio cis/trans).

### Example 3

Compound (V) as obtained in example 2, was further purified by slurring the obtained solid in a boiling solvent or by recrystallization. The results are summarized in the following table:

| | | | | Purity of the |
|---|---|---|---|---|
| | | | | isomeric mixture |
| solvent | type | volume | yield | of compound (V) |
| methanol | slurry | 1 g/15 mL | 70% | 94.2% |
| ethanol | slurry | 1 g/15 mL | 85% | 92.5% |
| Isopropanol | crystallization | 1 g/10 mL | 85% | 92.5% |
| n-butanol | crystallization | 1 g/10 mL | 70% | 93.5% |
| acetonitrile | crystallization | 1 g/15 mL | 80% | 95.5% |
| ethyl acetate | crystallization | 1 g/10 mL | 60% | 95.8% |
| acetone | crystallization | 1 g/10 mL | 50% | 94.2% |

### Example 4

Pure cis and trans isomers of compound (V) were isolated by chromatographic separation.

Cis-2-[4-(4-chlorophenyl)cyclohexyl]-3-(2-pyridin-2-ylthio)-naphthalene-1,4-dione, the cis isomer of compound (V): ¹³C-NMR (CDCl₃) δ 25.0, 30.3, 35.6, 42.9, 120.6, 122.8, 126.7, 126.8, 128.3, 129.3, 131.1, 132.5, 132.7, 133.4, 133.6, 136.7, 143.4, 149.8, 157.1, 157.7, 180.5, 183.1. IR (cm⁻¹) 3429, 1668, 1577, 1280. EI-MS 459 (M), 266 (M—C₁₂H₁₄Cl).

Trans-2-[4-(4-chlorophenyl)cyclohexyl]-3-(2-pyridin-2-ylthio)-naphthalene-1,4-dione, the trans isomer of compound (V): ¹³C-NMR (CDCl₃) δ 29.7, 34.3, 48.0, 120.6, 122.9, 126.7, 126.8, 128.1, 128.4, 131.5, 132.5, 132.8, 133.4, 133.6, 136.7, 143.7, 149.8, 157.7, 157.1, 145.8, 183.2, 180.5. IR (cm⁻¹) 2941, 1672, 1650, 1575, 1284. EI-MS 459 (M), 266 (M—C₁₂H₁₄Cl).

### Example 5

Compound (V) obtained as prepared in example 2 (2 g, 4.3 mmol, 48:38 ratio cis/trans), was admixed with methanol (40 mL) at 45° C., then NaOH (1.04 g, 0.026 mol) in water (7 mL) was added dropwise at a period of 10 minutes. After stirring for 0.5 h at 55-60° C., the mixture was extracted with heptane ×2 (10 mL), the phases were separated and then concentrated HCl (2 mL) was added to the aqueous phase. The resulting solid was filtered, washed with water, dried at 40° C. and recrystallized from acetonitrile to give compound (I) (atovaquone) (99.35% purity, 12% yield).

### Example 6

Compound (V) obtained as prepared in example 2 (5 g, 10.9 mmol, 48:38 ratio cis/trans), was admixed with methanol at 45° C., then K₃PO₄×3H₂O (8.8 g, 43 mmol) in water (25 mL) was added dropwise within ten minutes. After stirring for two hours at 50-55° C., the mixture was filtered and the filtrate was extracted with heptane twice (×15 mL). The phases were separated and the aqueous layer was acidified with concentrated HCl to pH=4-5. The resulting solid was filtered, washed with water and dried at 40° C. to give 2-[4-(4-chlorophenyl)cyclohexyl]-3-hydroxy-1,4-naphthoquinone (3.2 g, 48:41.5 ratio cis/trans, 80% yield).

1. A process for preparing atovaquone (compound I) comprising:
a) reacting 4-(4-chlorophenyl)cyclohexane-1-carboxylic acid of formula (II) with N-hydroxypyridine-2-thione of formula (III) in the presence of an esterification reagent, to form 2-thioxopyridin-1(2H)-yl-4-(4-chlorophenyl)-cyclohexane carboxylate, the compound of formula (IV); 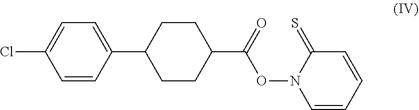
b) reacting compound (IV) with 1,4-napthoquinone to form 2-[4-(4-chlorophenyl)cyclohexyl]-3-(2-pyridin-2-ylthio)-naphthalene-1,4-dione, the compound of formula (V); 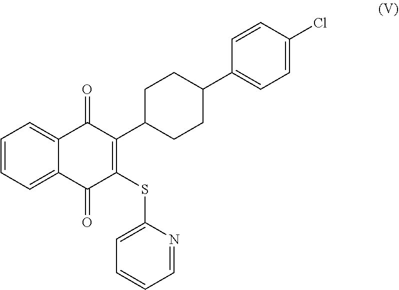
c) converting the compound of formula (V) into 2-[4-(4-chlorophenyl)cyclohexyl]-3-hydroxy-1,4-naphthoquinone of formula (VI) in the presence of a base; and
d) isolating the trans 2-[4-(4-Chlorophenyl)cyclohexyl]-3-hydroxy-1,4-naphthoquinone of formula (I).

2. The process of claim 1, wherein step (a) comprising:
admixing 4-(4-chlorophenyl)cyclohexane-1-carboxylic acid of formula (II) with N-hydroxypyridine-2-thione of formula (III) in an organic solvent,
cooling to reduce the temperature, adding an esterification reagent, optionally in several portions, and
isolating compound (IV).

3. The process of claim 2, wherein the organic solvent for the reaction of step (a) is selected from dichloromethane, dichloroethane, chloroform, acetonitrile, tetrahydrofuran (THF), acetone, dioxane or a mixture thereof.

4. The process of claim 3, wherein the organic solvent for the reaction of step (a) is dichloromethane

5. The process of claim 2, wherein the esterification reagent is selected from dicyclohexylcarbodiimide (DCC), 3-dimethylaminopropyl carbodiimide (EDC) and diisopropylcarbodiimide (DIC).

6. The process of claim 5, wherein the esterification reagent is dicyclohexylcarbodiimide (DCC).

7. The process of claim 2, wherein the reaction is carried out in a temperature range of −5° C. to 15° C.

8. The process of claim 7, wherein the reaction is carried out at 0-5° C.

9. The process of claim 1, wherein step (b) comprising:
irradiating compound (IV) with 1,4-napthoquinone in an organic solvent; and
isolating the obtained compound (V).

10. The process of claim 9, wherein the organic solvent for the reaction of step (b) is selected from dichloromethane, dichloroethane, chloroform, carbon tetrachloride, toluene, acetonitrile and mixture thereof.

11. The process of claim 10, wherein the organic solvent is dichloromethane.

12. The process of claim 1, wherein step (c) comprising:
reacting compound (V) with a base in a polar organic solvent at elevated temperatures.

13. The process of claim 12, wherein the polar organic solvent is selected from methanol, ethanol, 1-propanol, 2-propanol, N,N-dimethylformamide (DMF), N,N-dimethylacetamide (DMA), or mixture thereof.

14. The process of claim 13, wherein the polar organic solvent is methanol.

15. The process of claim 12, wherein the base is selected from sodium hydroxide, potassium hydroxide, lithium hydroxide, sodium carbonate, potassium phosphate, sodium phosphate and sodium bicarbonate.

16. The process of claim 15, wherein the base is sodium hydroxide.

17. The process of claim 12, wherein the reaction is carried at a temperature range of 50 to 65° C.

18. The process of claim 17, wherein the reaction is carried at 55-60° C.

19. The process of claim 1, wherein the isolation step (d) comprising:
collecting the solid obtained by filtration, washing, drying, and optionally recrystallizing the crude product.

20. A compound of formula (IV), salts or isomers thereof.

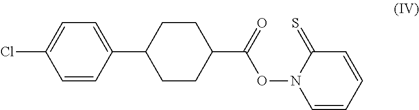

21. A compound of formula (V), salts or isomers thereof. [chemical structure]
